(19) United States
(12) Patent Application Publication
(10) Pub. No.: US 2006/0084169 A1
(43) Pub. Date: Apr. 20, 2006

(54) Polymer substrate for cultivating cells, in particular keratinozytes

(75) Inventors: Gregor Boese, Berlin (DE); Wolfgang Albrecht, Teltow (DE); Guenther Malsch, Teltow (DE); Thomas Groth, Berlin (DE); Andreas Lendlein, Berlin (DE); Hans-Jurgen Stark, Meckesheim (DE); Norbert Fusenig, Geesthacht (DE)

Correspondence Address: Norris, McLaughlin & Marcus P.A., 18th Floor, 875 Third Avenue, New York, NY, 10022, US

(73) Assignee: GKSS-Forschungszentrum Geesthacht GmbH, Geesthacht (DE)

(21) Appl. No.: 11/244,890

(22) Filed: Oct. 6, 2005

(30) Foreign Application Priority Data
Oct. 8, 2004 (DE) 10 2004 049 758.3

(51) Int. Cl. C12N 5/08

(52) U.S. Cl. 435/371; 435/404

(57) ABSTRACT

The invention relates to a polymer substrate for cultivating tissue cells. The substrate is provided in form of an oxygen-permeable flat membrane, preferably with ultra-filtration properties, and has an optimized amine content of at least 10 nmole amine function per cm² membrane. The substrate can be used to treat burns and other tissue defects. The substrate can be rapidly populated with cells, in particular with keratinozytes, and supports reorientation of the cells after the populated membrane is transferred to a wound or a dermis equivalent. The substrate can also be later easily detached from the newly formed tissue structure.

The invention relates to a polymer substrate and its use for cultivating cells, in particular keratinozytes. The invention also relates to an organotype hybrid structure with a polymer substrate and cells cultured on the substrate, as well as a method for producing a tissue structure, in particular an epidermal skin structure.

The therapy of chronic tissue defects, in particular of diabetic, venous and pressure-induced ulcerations as well as large burn wounds represents an important medical and economic problem. Approximately 2% of the world population suffers from wounds that have difficulty healing, resulting in significant overall treatment costs, with the number of cases steadily increasing. The treatment of chronic tissue defects and wounds through Tissue Engineering (TE), i.e., by using artificially cultured tissue structures, has shown early clinical success. The second important application of in vitro cultured skin substitutes is their use in dermatology for experimental purposes. They represent alternatives to expensive clinical studies or animal tests, so that wound healing, aging or skin irritation, which may be caused by physical or chemical influences, can be investigated with organotype test models generated through Tissue Engineering.

Purely biological skin replacement products, such as xeno-transplants, cadaver skin, and allogenous vital donor skin are only capable of temporarily covering the wound and are intended to stimulate the formation of a new dermal structure by induction of a granulation tissue. However, there is always a risk of infection and that the donor skin is rejected after some time due to immunological incompatibility, and hence must be removed before transplantation of split-skin (i.e., autogenous donor skin).

Also known for closing and covering a wound are hybrid skin replacement products, which contain a biologically derived component and a stable synthetic material. For example, U.S. Pat. No. 5,460,939 A discloses a three-dimensional support structure of a resorbable or non-resorbable material to be populated with cells on a semi-permeable membrane. Fibroblasts are cultured on the support structure in vitro for several weeks to produce a three-dimensional tissue network. This includes the product “Biobrane” (from the company Dow Hickam/Bertek Pharmaceuticals), which is made of a nylon fabric coated with pig collagen I and bonded to a silicone membrane. This is intended to improve adhesion in the wound bed and growth of fibroblasts and blood vessels. Another material used for treating burns (“Integra”, from the company Integra LifeScience Corp.) consists of a silicone membrane for covering the wound bed, with a bovine collagen network and glycose amino glucane anchored on its backside, intended to promote the regeneration of dermal structures (U.S. Pat. No. 5,489,304 A). In spite of positive clinical results, infections are sometimes observed which require a premature removal of the material. Another product uses autogenous skin cells which are embedded as a suspension in a fibrin adhesive and are applied to the wound bed as “skin out of the tube.” This approach is less suitable for treating large wounds.

In addition, TE products with predominantly allogenous cell and/or tissue structures are known. Commercially available TE products include “TransCyte” (from the company Advanced Tissue Science Inc.), which consists of a nylon fabric with immobilized neonatal allogenous fibroblasts, which undergo growth during an in vitro cultivation in excess of two weeks, producing extra-cellular matrix components. The cells are killed by cryo-conservation, leaving only the extra-cellular matrix. The matrix is populated after transplantation with fibroblasts to accelerate epithelialization of the wound. Also known is the dual-layer allogenous product “Apligraf” (from the company Organogenesis Inc.) which consists of a gel made of type I bovine collagen with living neonatal fibroblasts in a superpositioned epidermal layer of neonatal keratinozytes. This complex and expensive skin replacement products is used, in particular, to treat and promote the healing ulcers that have difficulty healing. The allogenous donor cells disappear after a certain time due to rejection reactions and are replaced by invading autogenous cells (Falanga V. et al., Arch Dermatol. 134(3), 1998, 293-300). Another product known under the name “Dermagraft” includes a cryo-conserved dermal structure consisting of neonatal allogenous fibroblasts, which are cultured on a resorbable polymer scaffold of polyglucogen. The fibroblasts grow in vitro until confluence occurs, during which time they secrete growth factors and extra-cellular matrix components, thereby forming a vital structure that can be transplanted (Hansbrough J. F. et al., Surgery 111(4), 1992, 438-446).

In summary, it can be safely stated that no products presently exist which adequately perform the functions of natural skin at moderate costs. Conventional cellular material include in particular allogenous cells, which are a major cause for rejection reactions. If non-resorbable polymers are used, these need to be removed, which not only adds to the medical expenses, but also causes bleeding. It is therefore an object of the present invention to provide a substrate, which forms a suitable base for in vitro cultivation of skin structures and can also be easily detached and removed from the treated wound, without the risk of injuring the newly formed skin structure.

The object is solved with a polymer substrate for cell cultures, which is provided in form of an oxygen-permeable flat membrane, with the membrane having an amine content of at least 10 nmole amine function per cm² membrane. It has been observed that air-permeable, aminated polymer materials in the form of porous membranes are excellent support structures for population with primary keratinozytes, if the amine content of the material is optimally adjusted either by modifying the membrane substance itself or the membrane surface, and if the membranes are porous enough to be sufficiently permeable to air. Advantageously, the substrate according to the invention initially promotes the proliferation of the keratinozytes and, due to a relatively weak adhesion of basal cells, facilitates the subsequent detachment of the membrane without harming the tissue.

These advantageous properties are achieved with an amine content of at least 50 nmole/cm², in particular of 100 to 550 nmole/cm², in particular from 200 to 500 nmole/cm², preferably from approximately 350 to 450 nmole amine function per cm² membrane. It is hereby unimportant if the amine content is the result of a substance modification or a surface modification. If the substance is modified, a polymer which has a corresponding amine functions (substance modification per se) or to which a polymer component with corresponding amine functions is added (volume modification) is used for fabricating the membrane. Conversely, if the surface is modified, the membrane surface is later provided with amine functions, i.e., either with a covalent surface functionalization through amine-containing groups (for example by wet-chemical modification or grafting) or with a non-covalent coating of the membrane with an amine-containing material. It would also be feasible to use a membrane which is both substance-aminated and surface-aminated. In the context of the present invention, the amine content referring to an area refers to the total molar content of amine functions per cm² of cut-out membrane, i.e., not only to the freely accessible amine groups on the surface. Amination is related to hydrophilization of the material surface, which can be characterized by measuring a contact angle.

In the context of the present invention, amine functions refers to any nitrogen-containing function, where nitrogen binds to hydrocarbon and/or hydrogen, in particular primary, secondary and tertiary amino groups (RNH₂, R¹—NH—R², or NR¹R²R³), primary and secondary imino groups (R═NH, or R¹═N—R²), quaternary ammonia groups (⁺NR¹R²R³R⁴), primary, secondary and tertiary amide groups (R—CO—NH₂, R¹—CO—NHR² or R¹—CO—NR²R³), and imide groups (R¹—CO—N═H²), as well as mixtures of thereof, wherein the moieties R, R¹, R² and R³ each represent a linear or branched, saturated or unsaturated, cyclic or acyclic hydrocarbon moiety. According to a preferred embodiment of the invention, the amine content includes, in particular, such nitrogen groups from the aforementioned group where the nitrogen has one or two hydrogen bonds. The tissue structures formed on the membrane surface of substrates aminated in this manner have a significantly reduced adhesion compared to those formed on other substrates, and more easily detach without harming the tissue. Preferably, the aforementioned optimized amine contents relate to primary and/or secondary amino groups (RNH₂, R¹—NH—R²).

According to a particular advantageous embodiment of the invention, the substrate is a membrane aminated by substance modification per se, which contains as a polymer material a co-polymer of acrylonitrile (AN) and an amine-function-containing co-monomer, in particular an aminoalkyl acrylamide or an aminoalkyl methacrylamide, wherein the alkyl group is a linear or branched, saturated or unsaturated C1-C5 alkyl group. A particularly preferred co-monomer is 3-amino propyl methacryl amide (APMA) or its hydrochloric adduct.

A particularly advantageous example for a surface functionalization is a membrane surface functionalized with oligomer or polymer, linear or branched amines, in particular polyalkyl imines, wherein the alkyl group is a linear or branched, saturated or unsaturated C1-C5 alkyl group. Particularly preferred is a polyethermide membrane aminated with polyethyleneimine(s).

In addition to the aforedescribed amine content, the substrate of the invention is also distinguished by its porosity, which ensures adequate gas transport and diffusion of metabolites through the membrane, but should also protect the treated wound from infections. This is achieved by using an ultra-filtration membrane which is impervious to microorganisms, in particular bacteria. This membrane has advantageous a pore size of at most 0.5 μm, in particular of at most 0.1 μm, and a water permeability of at least 0.5 l/m²hPa, in particular of at least 1.0 l/m²hPa.

For the population with cells, in particular keratinozytes, the membrane advantageously includes a plastic surface structure, which can be produced, for example, by subsequently embossing the membrane or already during the fabrication of the membrane. Such textured structure not only promotes the population of the surface, but can also induce an oriented tissue growth, which particularly well imitates the natural organo-mechanical properties of the skin.

According to another advantageous embodiment, the polymer used for the membrane is a material that cannot be biologically, in particular enzymatically, resorbed, whereby the material is completely detached and removed from the material after the transfer and the formation of a dense epidermal cell cover. This prevents complications that may result from decomposition products.

The invention also relates to the use of the aforedescribed polymer substrate for cultivating tissue cells, in particular for cultivating keratinozytes. In particular, the substrate can be used to produce an epidermal skin structure, which can be employed as skin transplant or as skin substitute for dermatological and pharmacological experiments. For producing epidermal skin transplants, autogenous cells removed from the patient are preferably used for cultivation.

Another aspect of the invention relates to an organotype hybrid structure, which includes the aforedescribed polymer substrate of the invention as well as tissue cells cultured on the substrate, in particular keratinozytes and/or an epidermal skin structure.

Another aspect of the invention relates to a method for producing a tissue structure, in particular an epidermal skin structure, with the steps of:
cultivating primary cells, in particular keratinozytes, during a first cultivation period on a polymer substrate according to one of the claims 1 to 14, wherein the substrate is provided in form of an oxygen-permeable flat membrane and the membrane has an amine content of at least 50 nmole amine per cm² membrane,
transferring the substrate, which is populated with the cells, to a target structure, so that the side of the substrate facing the cells rests on the target structure,
cultivating the cells for a second cultivation period, and
detaching and removing the substrate from the formed tissue structure (while leaving the tissue structure on the target structure).

In other words, the substrate of the invention is used to grow keratinozytes initially for a short time in vitro and to apply these keratinozytes as an epithelial layer to a target structure, for example to a dermis equivalent or a wound surface. This is done by bringing the cell side of the membrane into contact with the target structure (dermis equivalent, wound surface). The first cultivation is preferably performed only to a sub-confluent state of cells, i.e., without forming a closed cell layer. The cell culture experiments have shown that the aminated flat membranes of the invention facilitate rapid transmigration and repolarization (reorientation) of the keratinozytes, if the top surface of the membranes is placed on a culture surface, for example on cell culture dishes or collagen matrices, where they adhere and form multilayer epithelial structures. The formation of keratin layers on the surface, i.e., on the side facing the substrate, causes the support material to spontaneously detach, so that it can be easily removed. The method is particularly suited to apply keratinozytes over large wounds, where they can begin to grow while protected by the substrate material. The substrate material is spontaneously shed after the target substrate or the wound surface is covered. A small skin biopsy or a few cells from the root of a hair are already sufficient to obtain an almost closed epithelial cell cover of autologous cells within a relatively short time of, for example, several days. The ultra-filtration membranes are impervious to bacteria, but enable gas exchange required for a rapid stratification when the organotype culture is exposed to air. The flat membranes initially remain on the target structure as protection, to facilitate handling, and for the clinical application, whereafter they can be easily removed from the target structure without damaging the generated tissue.

Advantageous embodiments of the invention are recited as features of the dependent claims.

Exemplary embodiments of the invention will now be described with reference to the corresponding drawings.

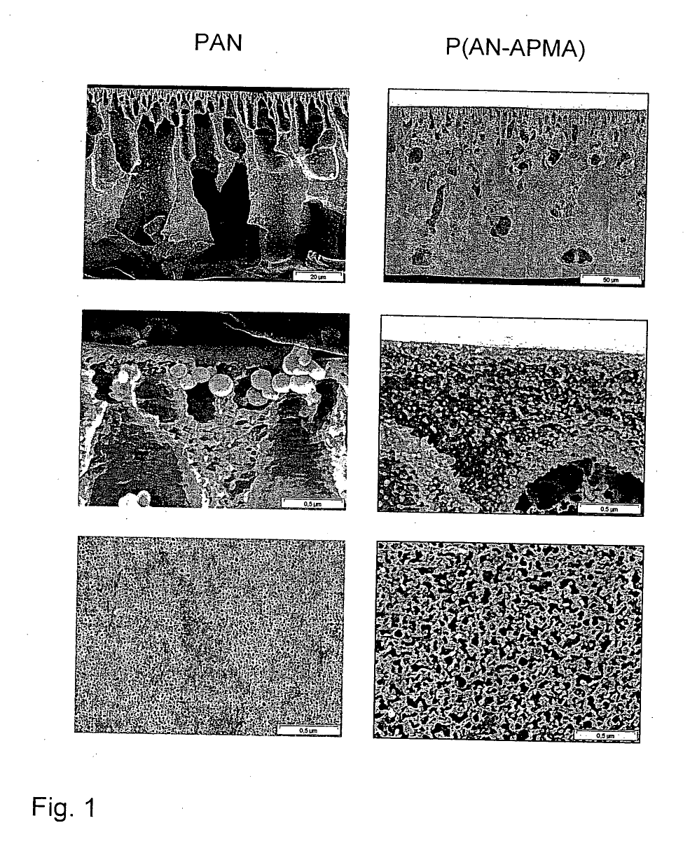

FIG. 1 shows REM images of cross-sections (upper and center row) and surfaces (lower row) of PAN and P(AN-APMA) membranes;

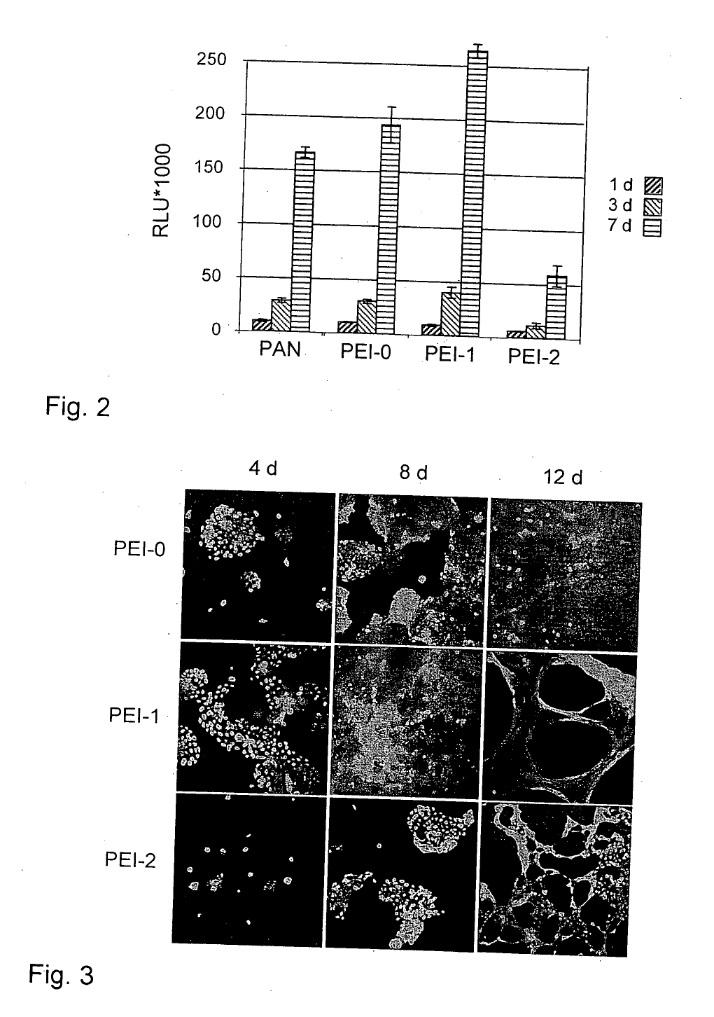

FIG. 2 shows results of an LDH assay for investigating the proliferation of HaCaT cells on PAN membranes as well as on unmodified, weakly modified, and strongly modified PEI membranes (PEI0, PEI1, and PEI2, respectively);

FIG. 3 shows scanned laser micrographs of vital staining of the HaCaT cells on unmodified, weakly modified, and strongly modified PEI membranes (PEI0, PEI1, and PEI2, respectively);

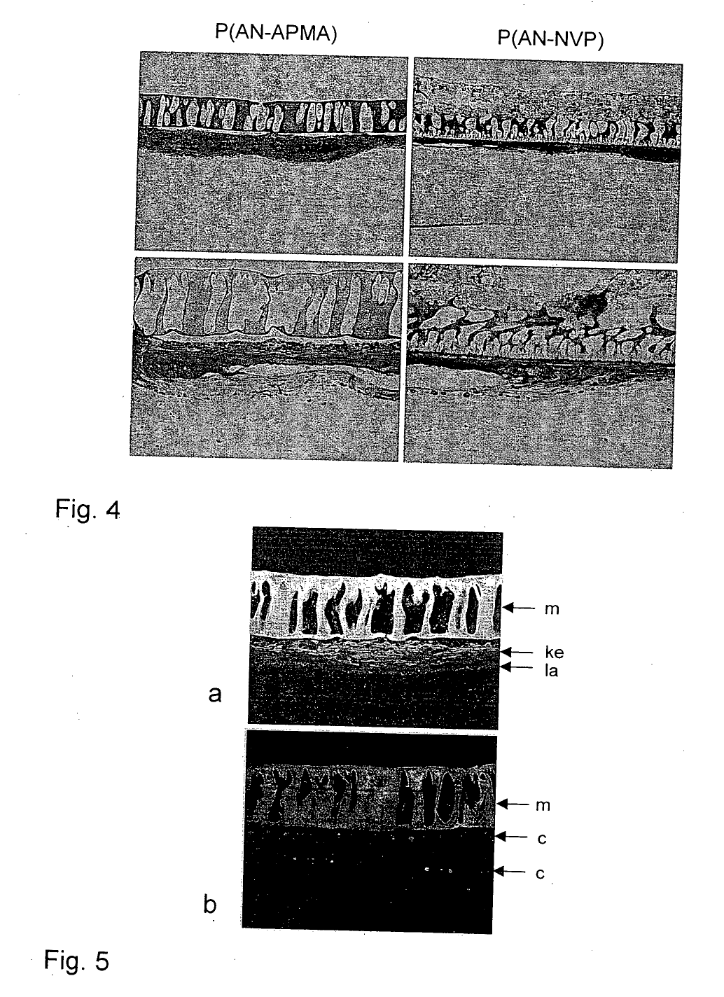

FIG. 4 shows micrographs of H&E staining of cryo-sections of organotype co-cultured keratinozytes on P(AN-APMA) and P(AN-NVP) membranes; and

FIG. 5 shows micrographs of immuno-fluorescence stains of the co-cultured keratinozytes on a P(AN-APMA) membrane.

### EXAMPLE 1

### Synthesis of PAN and P(AN-APMA)

The acrylonitrile polymers PAN (polyacrylonitril, homo-polymer) according to formula 1 as well as P(AN-APMA) (polyacrylonitrile-3-aminopropylmethacrylamide hydrochloride, copolymer) according to formula 2 were produced by radical-initiated solution polymerization of the

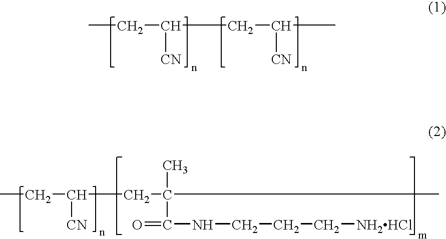

corresponding monomers and optionally copolymers (acrylonitrile and/or acrylonitrile and 3-amino propylmethacrylamide), precipitated in ethanol and dried. The chemical composition of the polymers was analyzed by element analysis, and the molar mass was determined by membrane osmometry. The results are summarized in Table 1.

TABLE 1
Polymer properties

| | X_{com} | M_{n, OBm} |
|---|---|---|
| Membrane | [Mole-%] | [10³ g/mole] |
| PAN | 0 | 48.5 |
| P(AN-APMA) | 7.1 | 55.7 |

X_{com} = Mole fraction of the co-monomers in the polymer, M_{n, Obm} = Molecular mass of the polymer.

Membranes were produced from the synthesized polymers and corresponding DMF polymer solutions by phase inversion technique under addition of water. The membranes were subsequently washed without a solvent and annealed. To improve handling, several of the membranes were pulled onto a support fabric (Histar TH100). The membranes were stored in an acidic solution in a refrigerator at 4° C. Table 2 summarizes characteristic data relating to the fabrication and the properties of the membranes. FIG. 1 shows raster electron-micrographs of the cross-sections (upper and center row) and surfaces (lower row) of the PAN and P(AN-APMA) membranes.

TABLE 2
Fabrication Parameters

| | c_{polymer} | vₚᵤₗₗ | Tₛₒₗ | Sedimentation | |
|---|---|---|---|---|---|
| Membrane | [wt.-%] | [m/min] | [° C.] | bath | Anneal |
| PAN | 15 | 4 | 1-2 | H₂O | 10 min |
| | | | | 5° C. | 90° C. |
| P(AN-APMA) | 15 | 2 | RT | H₂O | 10 min |
| | | | | RT | 90° C. |

c_{polymer} = concentration of the polymer in solution, vₚᵤₗₗ = pulling speed, Tₛₒₗ = temperature of the polymer solution.

### EXAMPLE 2

### Fabrication of Aminated PEI Membranes

Polyetherimide membranes (PEI membranes) according to formula 3 were aminated by surface functionalization of commercially available PEI membranes that have ultra-filtration properties with oligomer or polymer, linear or branched amines (in particular polyethyleneimine) using a process described by Albrecht et al. (Albrecht W., Seifert B, Weigel Th, Schossig M, Holländer A, Groth Th, Hilke R: Amination of polyetherimide membranes using di- and multivalent amines, Macromol. Chem. Phys. 2003, 204, 510-521). The carbonyl groups of the imide ring of the PEI membrane are covalently aminated by polyethyleneimine —[(CH₂)₂NH]ₙ— by forming an amide bond according to formula 4. The amide group is present as CONHR or as CONR¹R², depending if the reaction takes place with the terminal primary amino group or with a secondary amino group of the polyethyleneimine, wherein the moieties R, R¹, R² each represent a linear or branched polyethyleneimine moiety [(CH₂0₂NH]ₙ.

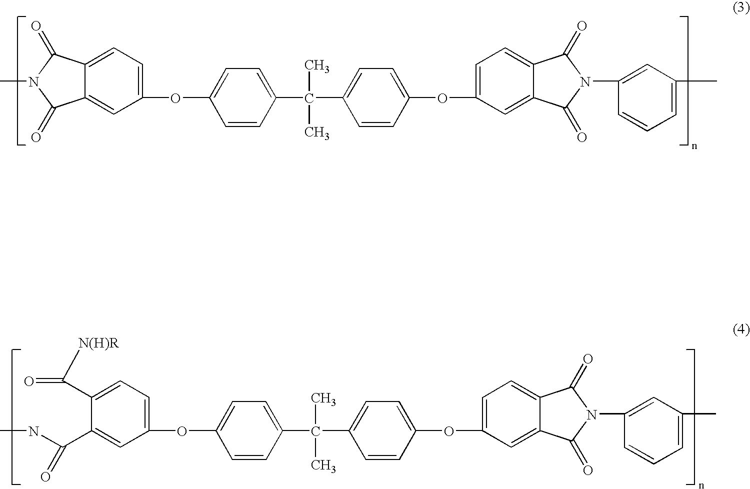

In a concrete example, PEI ultra-filtration membranes were aminated with a low molecular weight, linear polyethyleneimine with an average molecular weight of approximately 700 g/mole (PEI amine1), as well as with a high molecular weight, branched polyethyleneimine of approximately 60,000 g/mole (PEI amine2). PEI amine2 has a significantly higher amine content due to the high number of amine functions within each chain. Characteristic data of the aminated PEI membranes, which were surface-modified in this way, and of the substrate-modified PAN and P(AN-APMA) membranes produced according to example 1 are listed in Table 3. The amine content was determined with an Acid Orange test, which is described in detail in W. Albrecht et al. (see above).

TABLE 3
Membrane Properties

| | Water flow | Contact angle [°] | | Amine content |
|---|---|---|---|---|
| Membrane | [l/m²hkPa] | Wetting | Non-wetting | [nmol/cm²] |
| PAN | 4.02 | 59.5 | 43.0 | 0 |
| P(AN-APMA) | 0.70 | 81.3 | 48.4 | 400 |
| PEI | 3.86 | 79.5 | 48.5 | 0 |
| PEI-Amin1 | 3.5 | 61.8 | 33.8 | 410 |
| PEI-Amin2 | 0.7 | 55.8 | 13.2 | 650 |

### EXAMPLE 3

### Determination of the Toxicity of the Materials

Before the support membranes produced according to Examples 1 and 2 were used in cell population experiments, the toxicity of the materials was tested according to ISO 10993-5. Material extracts were produced by incubation with a cell culture medium over 1, 3, 7, and 14 days. 3T3 fibroblasts were cultured in 96 hole culture plates to pre-confluence, i.e., until the cells were arranged in the densest possible monolayer, and then covered for 24 hours with layers of the eluant. The metabolic activity and the membrane integrity of the cells were then determined by LDH or XTT assays and compared with control materials (3T3 fibroblasts cultured in a pure cell culture medium). No toxic effect was observed (results not shown).

### EXAMPLE 4

### Cultivation of Epidermal Skin Structures on Aminated Membranes

The keratinozyte cell line HaCaT was seeded on the polymer membranes according to the Examples 1 and 2 PAN, PEI (=PEI-0), PEI-Amin1 (=PEI-1) and PEI-Amin2 (=PEI-2). The activity of the cellular lactate dehydrogenase was determined after 1, 3, and 7 days incubation in an incubator through an LDH assay. The results are shown in FIG. 2. The membranes PAN and PEI-0 which were neither substance-aminated nor surface-aminated showed comparable activities. The “moderately” aminated PEI-membranes PEI-1 with an amine content of 410 nmole/cm² showed the highest enzyme activity after 7 days. Conversely, a significant reduction of the enzyme activity compared to the non-aminated membranes was detected in the strongly aminated membranes with an amine content of 650 nmole/cm².

These results were confirmed by vital staining of the HaCaT cells with fluorescene diacetate and subsequent evaluation in a laser scanning microscope (FIG. 3). The HaCaT cells on the weakly aminated PEI-1 membrane formed a closed cell cover after only 8 days. The results after 12 days on the PEI-1 membrane are noteworthy, because the cells become detached from the polymer membranes over large areas. However, LDH measurements have shown that the cells maintain a very high enzyme activity. This suggests that the aminated polymer membrane stimulates cell growth of keratinozytes, with the cells adhering only weakly to the material, which is desirable.

### EXAMPLE 5

### Cultivation of Epidermal Acrylnitrile Skin Structures on Copolymer Membranes

For these experiments, UF-membranes of P(AN-APMA) prepared according to Example 1 as well as a UF-membrane prepared in analogy to Example 1 from the copolymer polyacrylnitrile-N-vinyl-pyrrolidone (P(AN-NVP)) according to formula 5 were employed. Both materials have similar hydrophilic characteristics, as determined by contact angle measurements.

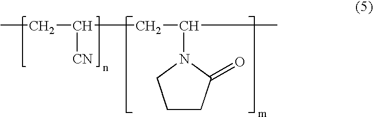

Primary keratinozytes were seeded on these UF-membranes and, after being cultured for 6 days, placed with their cell sides (“upside-down”) on a preformed standardized dermis equivalent, consisting of fibroblasts in a collagen gel. At this point, the cells almost completely cover the UF-membranes. The organotype co-cultures were histologically evaluated after 6 additional days in a serous medium while exposing the keratinozytes to air. Different results were obtained depending on the membrane material, as can be seen from the H&E staining of cryo-sections (upper and lower row: 10-fold and 20-fold enlargement, respectively). Only very thin neo-epidermis structures are formed under the P(AN-APMA) membranes (FIG. 4, right side), which strongly adhere to and remain connected to the material surface. Conversely, the significantly stronger formed epidermis under the P(AN-APMA) membrane (FIG. 4, left side) detached spontaneously almost completely from the aminated surface. In this way, the UF-membrane can advantageously be removed easily without damaging the newly formed tissue. The easier detachment of the skin structure from the P(AN-APMA) membrane is presumably related to the presence of primary and secondary amino groups and protons in the membrane material bonding to nitrogen, respectively.

The skin culture formed in vitro on the P(AN-APMA) membrane was investigated in detail by immuno-fluorescence microscope analysis. FIG. 5a shows the original green immuno-fluorescence color of keratin 1/10 (ke) and the original red fluorescence color of laminine (la). FIG. 5b shows an immuno-fluorescence color with the thymidine analogue bromide desoxyuridine (BrdU), where the original cell nuclei (c) show blue fluorescence. In both diagrams, the P(AN-APMA) membranes (m) exhibit auto-fluorescence. This investigation showed all the features of healthy skin. Differentiating keratinozytes reinforce the cell skeleton of the skin through synthesis of keratin 1 and keratin 10. The basal membrane with a large component of laminine-5 forms between dermis and epidermis. Surprisingly, proliferated, BrdU-positive keratinozytes were found not only next to the basal membrane, but also in the epithelial component which is in contact with the aminated UF-membrane (FIG. 5b). This two-sided polarization of the newly formed epithelial layer is caused by the fact that only a portion of the cells on the support material was already differentiated when they were transplanted to the dermis equivalent and were subsequently unable to reorient themselves. This can be prevented by shortening the initial growth time on the support material and through an early reorientation, i.e., by turning the support material over, so that the top surface of the grown keratinozytes come into contact early on with the wound bed that needs to be populated.

1. Polymer substrate for cultivating tissue cells, in particular for cultivating an epidermal skin structure, wherein the substrate is provided in form of an oxygen-permeable flat membrane, with the membrane having an amine content of at least 10 nmole amine function per cm membrane.

2. Polymer substrate according to claim 1, characterized in
in that the membrane has an amine content of at least 50, in particular of 100 to 550, in particular from 200 to 500, preferably from approximately 350 to 450 nmole amine function per cm² membrane.

3. Polymer substrate according to claims 1, characterized in that the amine content affects nitrogen functions, in which the nitrogen has at least one hydrogen bond, in particular primary and/or secondary amine functions.

4. Polymer substrate according to claim 1, characterized in
that the amine functions are present in the polymer substance of the membrane, in particular in the form of a substance modification or a volume modification.

5. Polymer substrate according to claim 4, characterized in
that the polymer substance comprises a copolymer of acrylonitrile (AN) and an amine-function-containing co-monomer.

6. Polymer substrate according to claim 5, characterized in
that the co-monomer is an aminoalkyl acrylamide or an aminoalkyl methacrylamide, wherein the alkyl group is a linear or branched, saturated or unsaturated C1-C5 alkyl group.

7. Polymer substrate according to claims 1, characterized in that the amine functions are present in form of a surface functionalization or a surface coating of the membrane.

8. Polymer substrate according to claim 7, characterized in
that the membrane surface is functionalized with oligomer or polymer amine functions, in particular with polyalkylimines, wherein the alkyl group is a linear or branched, saturated or unsaturated C1-C5 alkyl group.

9. Polymer substrate according to claim 7, characterized in that the membrane is a polyethermide membrane which is aminated with a polyethyleneimine.

10. Polymer substrate according to claim 1, characterized in
that the membrane is an ultra-filtration membrane which is impervious to microorganisms, in particular to bacteria.

11. Polymer substrate according to claim 1, characterized in
that the membrane has a pore size of at most 0.5 μm, in particular of at most 0.1 μm.

12. Polymer substrate according to claim 1, characterized in
that the membrane has a water permeability of at least 0.5 l/m²hPa, in particular of at least 1.0 l/m²hPa.

13. Polymer substrate according to claim 1, characterized in
that the membrane has a plastic surface structure.

14. Polymer substrate according to claim 1, characterized in
that the membrane is comprised of a non-resorbable polymer.

15. (canceled)

16. (canceled)

17. Organotype hybrid structure comprising a polymer substrate according to claim 1, as well as tissue cells cultured on the substrate, in particular an epidermal skin structure.

18. Method for producing a tissue structure, in particular an epidermal skin structure, with the steps of:
cultivating primary cells, in particular keratinozytes, during a first cultivation period on a polymer substrate according claims 1, wherein the substrate is provided in form of an oxygen-permeable flat membrane and the membrane has an amine content of at least 50 nmole amine per cm² membrane,
transferring the substrate, which is populated with the cells, to a target structure, so that the side of the substrate facing the cells rests on the target structure,
cultivating the cells for a second cultivation period, and
detaching and removing the substrate from the formed tissue structure.
